(19) United States
(12) Patent Application Publication
(10) Pub. No.: US 2024/0173281 A1
(43) Pub. Date: May 30, 2024

(54) TOPICAL COMPOSITIONS CONTAINING ANTHRANILIC ACID DERIVATIVES AND METHODS FOR TREATING SKIN DISORDERS

(71) Applicant: CHEMISTRY RX, FOLCROFT, PA (US)

(72) Inventor: Lars Brichta, Brooklyn, NY (US)

(73) Assignee: CHEMISTRY RX, FOLCROFT, PA (US)

(21) Appl. No.: 18/501,926

(22) Filed: Nov. 3, 2023

Related U.S. Application Data
(60) Provisional application No. 63/422,217, filed on Nov. 3, 2022.

(51) Int. Cl. A61K 31/196; A61K 9/00; A61K 9/06; A61K 47/06; A61K 47/20; A61K 47/26

(52) U.S. Cl. CPC A61K 31/196; A61K 9/0014; A61K 9/06; A61K 47/06; A61K 47/20; A61K 47/26

(57) ABSTRACT

Topical compositions containing anthranilic acid derivatives and a cream or an ointment base are described herein. Methods for treating skin conditions by administering the topical compositions are also described herein.

### CROSS-REFERENCE TO RELATED APPLICATIONS

The present application claims the benefit of U.S. Provisional Application No. 63/422,217, filed on Nov. 3, 2022, which is hereby incorporated in its entirety.

### TECHNICAL FIELD

The present disclosure relates to topical compositions containing anthranilic acid derivatives and methods for treating skin disorders.

### BACKGROUND

Disorders of the skin can be debilitating and painful for affected patients and substantially reduce the quality of life. Often, treatment modalities are very limited. Topical therapies are applied specifically to the affected skin areas. Such therapies are typically preferred over systemic treatments to reduce the exposure of the body to the respective drug, and thus reduce the potential for side effects. Topical preparations that contain anthranilic acid derivates in a cream or ointment are currently not available for the treatment of skin disorders in humans.

One such skin disorder that is particularly painful and debilitating is keratitis-ichthyosis-deafness (KID) syndrome. Kid syndrome is a rare genetic disorder caused by mutations in the Gap Junction Protein Beta 2 (GJB2) gene. GJB2 encodes for Connexin-26, a crucial structural protein that forms gap junction channels which connect neighboring cells and facilitate the exchange of certain molecules and ions. Manifestations of KID syndrome include cornea defects and other eye abnormalities which can lead to blindness, debilitating painful skin abnormalities, and hearing impairment. The onset of KID syndrome is typically at birth. Treatment options for all manifestations of KID syndrome are very limited, making the management of these patients extremely challenging. Novel, innovative treatment approaches are needed to better address the clinical needs of patients with KID and improve their quality of life. Therapeutic modalities that address the skin manifestations in patients with KID syndrome need to focus on single-drug topical preparations with limited inactive ingredients to ensure that the treatment is not only effective but also safe, without the risk of absorption of unwanted active or inactive ingredients through the skin. This is of particular importance given that the skin barrier in patients with KID is compromised, and most patients with KID are very young children.

### SUMMARY OF THE INVENTION

This disclosure pertains to a topical composition comprising anthranilic acid derivatives and methods for treating skin disorders.

A topical composition is described that includes an anthranilic acid derivative and a cream base or an ointment base. In certain aspects, the anthranilic acid derivative is mefenamic acid (N-(2,3-dimethylphenyl)anthranilic acid). In some aspects, the composition also includes at least one compound selected from the group consisting of a solvent, a penetration enhancer, a compound having both solvent and penetration enhancer properties, and a combination thereof. In some aspects, the anthranilic acid derivative is about 0.1% to about 15% (w/w) of the total composition. In some aspects, the at least one compound is about 0.5% to about 40% (w/w) of the total composition. In some aspects, the at least one compound may be dimethyl sulphoxide (DMSO), ethyl alcohol, diethylene glycol monoethyl ether, polyethylene glycol (PEG), benzyl alcohol, or hydrogenated castor oil. In certain aspects, the composition includes a cream base containing an oil-in-water emulsion or a water-in-oil emulsion. In other aspects, the composition includes an ointment base containing petrolatum. In some aspects, the composition may also include an emulsifying agent. In certain aspects, the composition contains an anthranilic acid derivative that is about 1% to about 3% (w/w) of mefenamic acid and may also contain about 5% to about 10% (w/w) dimethyl sulphoxide (DMSO).

This disclosure also pertains to a method for treating skin conditions by topically administering the composition described herein. In some aspects, the skin disorder is a connexin-related disorder. In certain aspects, the skin disorder is keratitis ichthyosis deafness (KID) syndrome.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, 2 μm, 3 μm, 4 μm, 5 μm, 6 μm, and 7 μm are also intended to be explicitly disclosed, as well as the range of values greater than or equal to 1 μm and the range of values less than or equal to 8 μm.

All percentages, parts and ratios are based upon the total weight of the topical compositions, unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55,” “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject, including applying to the skin.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic, or other agent across a tissue layer such as the stratum corneum, or stratum spinosum.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject or enhance the texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government or listed in the U.S. Pharmacopeia or other generally recognized pharmacopeia for use in mammals (e.g. animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, aryl aliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylate, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethane sulfonic, sulfanilic, cyclohexyl aminosulfonic, alginic, 3-hydroxybutyric, galactaric and galacturonic acid.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “treating” is used herein, for instance, in reference to methods of treating a skin disorder or a systemic condition, and generally includes the administration of a compound or composition which reduces the frequency of, or delays the onset of, symptoms of a medical condition or enhance the texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various embodiments are directed to topical compositions containing one or more anthranilic acid derivatives for treating skin conditions, such as growths and skin abnormalities, and in certain embodiments, the skin condition may be associated with a connexin-related disorder, for example and without limitation keratitis ichthyosis deafness (KID) syndrome. Other embodiments are directed to methods for treating skin conditions that include topically administering a composition containing one or more one anthranilic acid derivatives to a subject in need of treatment.

Anthranilic acid is an aromatic acid having the formula C6H4(NH2)(C02H) consisting of a benzene ring, ortho-substituted with a carboxylic acid and an amine. Anthranilic acid is commonly used as an intermediate in the production of azo dyes and saccharin. Fenamic acid is a derivative of anthranilic acid that is a nitrogen isostere of salicylic acid. Several compounds derived from fenamic acid or anthranilic acid, referred to herein as “anthranilic acid derivatives,” are used as non-steroidal anti-inflammatory drugs, including, for example, mefenamic acid, tolfenamic acid, flufenamic acid, and meclofenamic acid. The compositions and methods of embodiments can include any one or various combinations of these anthranilic acid derivatives.

In some embodiments, the anthranilic acid derivative may be mefenamic acid (N-(2,3-dimethylphenyl)anthranilic acid). The chemical structure of mefenamic acid is shown below as Structure (I);

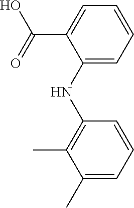 [SMILES: Cc1cccc(Nc2ccccc2C(=O)O)c1C]

Methods of producing anthranilic acid derivatives including mefenamic acid are well known. For example, methods for making anthranilic acid derivatives including mefenamic acid are U.S. Pat. Nos. 3,138,636 and 4,135,050, which are hereby incorporated by reference in their entirety.

The concentration of anthranilic acid derivatives in the compositions of embodiments can be up to about 15% (w/w) of one or more anthranilic acid derivatives. For example, in some embodiments, the composition may include about 0.1% (w/w) to about 15% (w/w), about 0.5% (w/w) to about 10% (w/w), about 0.75% (w/w) to about 7.5% (w/w), about 1% (w/w) to about 5% (w/w), about 1% (w/w) to about 3% (w/w), or any range or individual concentration of anthranilic acid derivatives encompassed by these example ranges. In particular embodiments, the composition may include about 0.25% (w/w) to about 5% (w/w) anthranilic acid derivatives, and in some embodiments, the compositions may include about 0.25% (w/w) to about 5% (w/w) mefenamic acid or derivatives thereof.

In embodiments, the compositions may include at least one of a solvent and/or a penetration enhancer. In some embodiments the solvent may also be a penetration enhancer and may be used interchangeably herein. For example, in some embodiments, the solvent and/or penetration enhancer may be monoethyl ether, ethyl alcohol, benzyl alcohol, or hydrogenated castor oil, a sulphoxide, for example, dimethyl sulphoxide (DMSO), decyl methyl sulfoxide, azone (1-dodecyl acyclic heptan-2-one or laurocapram), pyrrolidones such as N-methyl pyrrolidone, 2-pyrrolidone and N-cyclohexyl-2-pyrrolidone, and mixtures thereof, oxazolidinones, and urea. In some embodiments, the solvent and/or penetration enhancers may be a fatty alcohol such as cetyl alcohol, stearyl alcohol, decanol, tridecanol, lauryl alcohol, linolenyl alcohol, 2-(2-ethoxyethoxy)ethanol, or oleyl alcohol, and mixtures thereof or a fatty acid ester such as glycerol monolaurate, glycerol monooleate, glycerol monolinoleate, isopropyl isostearate, isopropyl palmitate, isopropyl myristate, diethyl sebacate, sorbitan monopalmitate, sorbitan oleate, sorbitan dilaurate, sorbitan trioleate, propylene glycol monolaurate and sucrose monolaurate, and mixtures thereof. In further embodiments, the solvent and/or penetration enhancer may be diols such as 1,2-hexanediol, butylene glycol, diethylene glycol, dipropylene glycol, ethyl hexanediol, ethylene glycol, hexylene glycol, pentylene glycol, propylene glycol, propylene glycol monolaurate, tetraethylene glycol, triethylene glycol, tripropylene glycol, polyethylene glycol (PEG), and polypropylene glycol, and mixtures thereof or polyols such as butanetriol, glycerol and 1,2,6-hexanetriol, and mixtures thereof. In some embodiments, the penetration enhancer may be a carrier and vehicle such as microcapsules or nanocapsules, nanoemulsions, submicron emulsions, miniemulsions, solid lipid nanoparticles, multiple emulsions, microemulsions, liposomes, niosomes, transfersomes (i.e. vesicles composed of phospholipids and 10% to 25% surfactant such as sodium cholate and 3% to 10% ethanol), ethosomes, aquasomes, and the like and combinations thereof. In still other embodiments, the solvent and/or penetration enhancer may be fatty acids such as essential oil, terpenes, terpenoids, oleic acid, capric acid, hexanoic acid, lauric acid, linoleic acid, linolenic acid, propionic acid and vaccenic acid, and mixtures thereof. In further embodiments, the penetration enhancer may be a surfactant. In still further embodiments the solvent and/or penetration enhancer may be a combination of one or more classes.

The compositions of the various embodiments described herein may include at least one compound selected from the group consisting of a solvent, a penetration enhancer, a compound having both solvent and penetration enhancer properties, and a combination thereof. The at least one compound in the compositions of various embodiments described herein may be present in an amount about 0.5% (w/w) to about 40% (w/w), about 1% (w/w) to about 20% (w/w), about 5% (w/w) to about 15% (w/w), or any range or individual concentration encompassed by these example ranges based on the total composition. In an example embodiment, DMSO may function as both a solvent and a penetration enhancer for a composition containing mefenamic acid.

The compositions of various embodiments can be in any form, including creams, lotions, foams, liniments, balms, ointments, soaps, shampoos, and the like.

Creams refer to semi-solid emulsions of oil and water in approximately equal proportions and may contain more than 20% water and volatiles. They may be divided into two types: oil-in-water (O/W) emulsions, composed of small droplets of oil dispersed in a continuous phase; and water-in-oil (W/O) emulsions, composed of small droplets of water dispersed in a continuous oily phase. Creams can provide a barrier to protect the skin. This may be a physical barrier or a chemical barrier as with UV-absorbing compounds. To aid in the retention of moisture (especially water-in-oil creams), creams are usually used for a variety of purposes including cleansing, emollient effects, and as a vehicle for drug substances such as local anesthetics, anti-inflammatoires (other NSAIDs or corticosteroids), hormones, antibiotics, antifungals or counterirritants. Creams also include emollient creams, transdermal creams, and gels.

Lotions are low- to medium-viscosity topical preparation. Most lotions are oil-in-water emulsions containing an emulsifier such as cetyl alcohol to prevent separation of these two phases. Lotions can include fragrances, glycerol, petroleum jelly, dyes, preservatives, proteins and stabilizing agents.

Pharmaceutical foams are pressurized dosage forms containing one or more active ingredients that, upon valve actuation, emit a fine dispersion of liquid and/or solid materials in a gaseous medium. Foam formulations are generally easier to apply, are less dense, and spread more easily than other topical dosage forms. Foams may be formulated in various ways to provide emollient or drying functions to the skin, depending on the formulation constituents. Accordingly, this delivery technology is a useful addition to the spectrum of formulations available for topical use.

Liniments or balms are topical formulations that are of a similar viscosity to lotions and less viscous than an ointment or cream. Liniments are generally applied with friction by rubbing the liniment into the skin. Liniments typically are formulated from alcohol, acetone, or similar quickly evaporating solvents and may contain counterirritant aromatic chemical compounds such as methyl salicylate, benzoin resin, or capsaicin.

An ointment is a mostly water-free vehicle for topical application to the skin. Ointments may be compositions in which oil and water are provided in a ratio of from 7:1 to 2:1, from 5:1 to 3:1, or 4:1 and may contain less than 20% water and volatiles. Ointments are generally formulated using oils, waxes, water, alcohols, petroleum products, water, and other agents to prepare formulations with various viscosities and solvent properties. Commonly used formulations include petrolatum, oleaginous base (e.g., white ointment), absorption base, W/O emulsion base (e.g., cold cream type base), O/W emulsion base (e.g., hydrophilic ointment), water soluble base, in addition to others. These preparations are used to dissolve or suspend substances or products with medicinal or cosmetic value.

In some embodiments, the formulations can be in the form of a soap, which are formulations that comprise a salt of a fatty acid. Soaps are mainly used as surfactants for washing, bathing, and cleaning, but they are also used in textile spinning and are important components of lubricants. Soaps for cleansing are usually obtained by treating vegetable or animal oils and fats with a strongly alkaline solution. Fats and oils are composed of triglycerides; three molecules of fatty acids are attached to a single molecule of glycerol. The alkaline solution, which is often called lye (although the term “lye soap” refers almost exclusively to soaps made with sodium hydroxide), is believed to promote a chemical reaction known as saponification. In saponification, the fats are first hydrolyzed into free fatty acids, which then combine with the alkali to form crude soap. Glycerol (glycerine) is usually liberated and is either left in or washed out and recovered as a useful byproduct, depending on the process employed.

In certain embodiments, the compositions may include a base such as, for example, a cream base (e.g., oil-in-water emulsion, water-in-oil emulsion, emollient, transdermal, gel, etc.), an ointment base (e.g., petrolatum, etc.), white petrolatum, white petrolatum USP, mineral jelly, petroleum jelly, yellow petrolatum, yellow soft paraffin, white soft paraffin, fats, waxes, sterols, fat-soluble vitamins, monoglycerides, diglycerides, triglycerides, phospholipids, and the like and combinations thereof. An example, of white petrolatum, but not limited to, is Aquaphor® ointment. In some embodiments, the base may be a liposomal base. Liposomal bases are an emulsion that includes a lipophilic component and an aqueous component that can be in the form of a lotion, a cream, a gel, or a paste. Examples of suitable liposomal bases include PCCA Lipoderm®, Lipoderm ActiveMax™, Anhydrous Lipoderm, and Lipoderm High Molecular Weight™ PCCA. Such liposomal base formulations can include, for example, about 60-80% w/w water combined with glycerin, C12-1s alkyl benzoate, glyceryl stearate, dimethicone, cetearyl alcohol, cetearyl glucoside, polyacrylamide, cetyl alcohol, magnesium aluminum silicate, xanthan gum, aloe vera (aloe barbadensis), tocopheryl acetate (vitamin E acetate), Grunus amygdalus amara (bitter almond) kernel oil, Vitis vinifera (Grape) seed extract, Triticum vulgare (wheat) germ oil, retinyl palmitate (vitamin A palmitate), ascorbyl palmitate (vitamin C palmitate), Pro-Lipo Multi-emulsion Liposomic System, tetrasodium EDTA, phenoxyethanol, sodium hydroxymethylglycinate and the like and combinations thereof. In particular embodiments, the base may be petrolatum, hydrophilic petrolatum, hydroxystearic sulfate, or anhydrous lanolin.

The amount of base in the compositions of embodiments can vary and will depend on the amounts of the other components. More base can be added to compensate for smaller amounts of other components in the desired topical pharmaceutical formulation. In some embodiments, the base may be present in a concentration of about 65% (w/w) to about 99% (w/w) of the total composition, or any range or individual concentration known in the art.

In some embodiments, the compositions may include an antioxidant. Such antioxidant may be, for example, butylated hydroxytoluene, ascorbic acid, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and the like and pharmaceutically acceptable salt or ester thereof or combinations thereof. The antioxidant can be present in a concentration of about 0.01% (w/w) to about 1% (w/w) of the total composition or any individual concentration encompassed by this example range.

In some embodiments, the composition may include an emulsifying agent, or emulsifier, including, for example, various polysorbates, monoglycerides, diglycerides, triglycerides, and blends thereof at a concentration of about 1% (w/w) to about 10% (w/w) of the total composition, or any range or individual concentration encompassed by this range. For example, without limitation, the range may be about 1% (w/w) to about 3% (w/w). In an example, the emulsifying agent, or emulsifier, may be sorbitan monooleate. In particular embodiments, compositions formulated as ointments may require an emulsifying agent to incorporate the solvent, such as, for example, DMSO.

In some embodiments, the compositions may further include a humectant that provides soothing, smoothing, moisturizing, or protects the skin. The humectant is not limited and can be, for example, calamine, dodecyl sulphate, sodium lauryl sulphate (SLS), a polyoxyethylene ester of polysorbitan, such as monooleate, monolaurate, monopalmitate, monostearate esters, esters of sorbitan, the polyoxyethylenes ethers, the sodium dioctyl sulfosuccinate (DOSS), lecithin, and sodium docusate. The amount of humectant in such compositions may be about 0.01% (w/w) to 5% (w/w) of the total composition.

In some embodiments, the composition may further include a UV-absorbing compound such as, for example, glyceryl PABA, padimate 0, roxadimate, dioxybenzone, oxybenzone, sulisobenzone, octocrylene, octyl methoxycinnamate, ethoxyethyl p-methoxycinnamate, homomenthyl salicylate, ethylhexyl salicylate, trolamine salicylate, avobenzone, ecamsule, ensulizole, bemotrizinol, bisoctrizole, and the like and combinations thereof. The amount of UV-absorbing compound may be about 0.01% (w/w) to 5% (w/w) of the total composition.

In some embodiments, the composition may further include an analgesic agent such as, for example, methyl salicylate, codeine, morphine, methadone, pethidine, buprenorphine, hydromorphine, levorphanol, oxycodone, fentanyl, a non-steroidal anti-inflammatory drug (NSAID), and the like and combinations thereof. The amount of the analgesic agent such compositions may be about 0.01% (w/w) to 5% (w/w) of the total composition.

Particular compositions encompassed by the various embodiments described above include about 0.5% (w/w) to about 5% (w/w) mefenamic acid or a derivative thereof, DMSO, and a cream base or ointment base. The ointment base may be petrolatum, such as, for example, hydrophilic petrolatum. In some embodiments, the compositions may include about 0.5% (w/w) to about 5% (w/w) mefenamic acid or a derivative thereof, about 5% (w/w) to about 25% (w/w) DMSO, and a cream base or ointment base, and in particular embodiments, the compositions may include about 2% (w/w) to about 4% (w/w) mefenamic acid or a derivative thereof, about 5% (w/w) to about 10% (w/w) DMSO and a cream or ointment base. In certain embodiments including an ointment base, an emulsifying agent, such as, for example, sorbitan monooleate may also be required. In an example embodiment, the composition may include about 2% (w/w) mefenamic acid, about 5% (w/w) DMSO, and about 93% (w/w) of a cream base. In another example embodiment, the composition may include about 2% (w/w) mefenamic acid, about 5% (w/w) DMSO, about 90% (w/w) of an ointment base, such as petrolatum, and about 3% of an emulsifying agent, such as sorbitan monooleate.

Other embodiments of the invention include methods for treating skin conditions, including growths and skin abnormalities by topically administering any of the compositions described above. Such methods are not limited to particular indications; however, the compositions described herein can be particularly useful for treating skin conditions associated connexin-related disorders, such as keratitis ichthyosis deafness (“KID”) syndrome. Keratitis ichthyosis deafness syndrome is a rare, genetic, multi-system disorder that is characterized by defects of the surface of the corneas (keratitis), red, rough thickened plaques of skin (erythrokeratoderma), and sensorineural deafness or severe hearing impairment. KID syndrome belongs to a group of skin disorders marked by dry, scaly skin known as the ichthyoses, and the skin on the palms of the hands and soles of the feet and the nails may be affected.

The methods of such embodiments may include the steps of administering a composition of the various embodiments described above to the skin of subject in need of treatment topically by, for example, applying the composition to affected areas such as the scalp or skin. The step of administering can be carried out one, two, three, four, or more times per day, and administering can be carried out the prescribed number of times per day for one or more days, one or more weeks, one or more months, one or more years or indefinitely. In some embodiments, administering can be carried out until the symptoms associated with the underlying conditions have been reduced or eliminated.

The step of administering can be carried out by various means. For example, administering can be accomplished by applying the composition to the skin of a subject, and in some embodiments, the skin may be massaged or rubbed to facilitate uptake of the active ingredients.

As used herein, “including”, “containing” and like terms are understood in the context of this application to be synonymous with “comprising” and are therefore open-ended and do not exclude the presence of additional undescribed and/or unrecited elements, materials, ingredients and/or method steps.

As used herein, “consisting of” is understood in the context of this application to exclude the presence of any unspecified element, ingredient and/or method step.

As used herein, “consisting essentially of” is understood in the context of this application to include the specified elements, materials, ingredients and/or method steps and others that are unspecified, provided that the latter “do not materially affect the basic and novel characteristic(s)” of what is being described.

The compositions and methods are further supported by the information provided in the following Examples. It is to be understood that the embodiments described in the Examples are merely illustrative and are not intended to limit the scope of the present disclosure, which will be limited only by the appended claims.

### EXAMPLES

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

### Example 1: Cream Composition

A topical composition formulated as a cream is provided in Table 1:

TABLE 1

| Ingredient | Amount |
|---|---|
| Mefenamic acid | 3% |
| DMSO | 10% |
| Cream base | 87% |

The composition of Table 1 was formulated as a cream by dissolving the mefenamic acid in DMSO and mixing in a cream base. The topical cream composition was formulated for administration by applying, such as by rubbing, directly on the skin of a patient diagnosed with keratitis ichthyosis deafness (KID) syndrome for symptoms including dryness and scaling. The composition was administered at least once daily for as long as symptoms persisted or as needed.

### Example 2: Ointment Composition

A topical composition formulated as an ointment is provided in Table 2:

TABLE 2

| Ingredient | Amount |
|---|---|
| Mefenamic acid | 2% |
| DMSO | 5% |
| Sorbitan Monooleate | 2.5% |
| Ointment base | 90.5% |

The is an example in which mefenamic acid was formulated as a topical ointment composition, which is sometimes needed for irritated, very sensitive, and/or dry skin. The composition included about 2% (w/w) of mefenamic acid, about 5% (w/w) of DMSO as a solvent and also a penetration enhancer, and about 2.5% (w/w) sorbitan monooleate as an emulsifier, and about 90.5% (w/w) of an ointment base, e.g., petrolatum. An ointment composition may be preferred in some cases due to an expected longer shelf-life and higher stability in comparison to preparations with water or creams. The topical ointment composition was administered directly to the skin at least once daily as needed or as long as symptoms persist.

A patient having KID syndrome was treated with the composition of Table 2 by applying the ointment once daily to the affected areas of the skin.

Whereas particular features of the present invention have been described above for purposes of illustration, it will be evident to those skilled in the art that numerous variations of the details of the compositions and methods disclosed herein may be made without departing from the scope in the appended claims.

What is claimed is:

1. A topical composition comprising an anthranilic acid derivative and a cream base or an ointment base.

2. The topical composition of claim 1, wherein the anthranilic acid derivative comprises mefenamic acid (N-(2,3-dimethylphenyl)anthranilic acid).

3. The topical composition of claim 1, further comprising at least one compound selected from the group consisting of a solvent, a penetration enhancer, a compound having both solvent and penetration enhancer properties, and a combination thereof.

4. The topical composition of claim 1, wherein the anthranilic acid derivative comprises about 0.1% to about 15% (w/w) of the total composition.

5. The topical composition of claim 3, wherein the at least one compound comprises about 0.5% to about 40% (w/w) of the total composition.

6. The topical composition of claim 3, wherein the at least one compound comprises dimethyl sulphoxide (DMSO), ethyl alcohol, diethylene glycol monoethyl ether, polyethylene glycol (PEG), benzyl alcohol, or hydrogenated castor oil.

7. The topical composition of claim 1, wherein the cream base comprises an oil-in-water emulsion or a water-in-oil emulsion.

8. The topical composition of claim 1, wherein the ointment base comprises petrolatum.

9. The topical composition of claim 1, further comprising an emulsifying agent.

10. The topical composition of claim 1, wherein the anthranilic acid derivative is about 1% to about 3% (w/w) of mefenamic acid.

11. The topical composition of claim 10, further comprising about 5% to about 10% (w/w) dimethyl sulphoxide (DMSO).

12. A method for treating a skin condition comprising topically administering to a subject in need of treatment a topical composition comprising an anthranilic acid derivative and a cream base or an ointment base.

13. The method of claim 12, wherein the topical composition comprises about 0.1% to about 15% (w/w) of the anthranilic acid derivative.

14. The method of claim 12, wherein the anthranilic acid derivative comprises mefenamic acid (N-(2,3-dimethylphenyl) anthranilic acid).

15. The method of claim 12, wherein the composition further comprises at least one compound selected from the group consisting of a solvent, a penetration enhancer, a compound having both solvent and penetration enhancer properties, and a combination thereof.

16. The method of claim 15, wherein the at least one compound comprises about 0.5% (w/w) to about 40% (w/w) of the total composition.

17. The method of claim 15, wherein the at least one compound comprises dimethyl sulphoxide (DMSO), ethyl alcohol, diethylene glycol monethyl ether, polyethylene glycol (PEG), benzyl alcohol, or hydrogenated castor oil.

18. The method of claim 12, wherein the cream base comprises an oil-in-water emulsion or a water-in-oil emulsion.

19. The method of claim 12, wherein the ointment base comprises petrolatum.

20. The method of claim 12, wherein the composition further comprises an emulsifying agent.

21. The method of claim 12, wherein the anthranilic acid derivative is about 1% to about 3% (w/w) of mefenamic acid.

22. The method of claim 21, wherein the composition further comprises about 5% to about 10% (w/w) of dimethyl sulphoxide (DMSO).

23. The method of claim 12, wherein the skin condition is a connexin-related disorder.

24. The method of claim 23, wherein the connexin-related disorder is keratitis ichthyosis deafness (KID) syndrome.
